(19) United States
(12) Patent Application Publication
(10) Pub. No.: US 2023/0165781 A1
(43) Pub. Date: Jun. 1, 2023

(54) COMPOSITIONS FOR TREATING DENTAL WHITE SPOTS

(71) Applicant: The Regents of the University of California, Oakland, CA (US)

(72) Inventors: Alireza MOSHAVERINIA, Los Angeles, CA (US); Sahar ANSARI, Los Angeles, CA (US); Benjamin M. WU, Los Angeles, CA (US); Mohammad Mahdi HASANI-SADRABADI, Los Angeles, CA (US)

(73) Assignee: The Regents of the University of California, Oakland, CA (US)

(21) Appl. No.: 17/925,685

(22) PCT Filed: May 20, 2021
(86) PCT No.: PCT/US2021/033370

§ 371 Date: Nov. 16, 2022

Related U.S. Application Data
(60) Provisional application No. 63/027,679, filed on May 20, 2020.

(51) Int. Cl. A61K 8/81; A61K 8/29; A61K 8/25; A61Q 11/00; A61K 8/04; A61K 8/73

(52) U.S. Cl. CPC A61K 8/817; A61K 8/25; A61K 8/29; A61K 8/042; A61K 8/733; A61Q 11/00; A61K 2800/614; A61K 2800/651

(57) ABSTRACT

The present disclosure provides bioactive dental restorative materials and methods for making and using them. In one embodiment, the invention relates to the development of novel poly dopamine (PDA) nanoparticles with remineralization capability.

### FIELD OF THE INVENTION

The present invention relates in general to dental compositions and methods of making and using the same for treatment of dental white spots.

### BACKGROUND

In general, white spots on teeth are primarily caused by the process of decalcification or demineralization (early caries) underneath intact dental enamel. During this process, acid produced by bacteria dissolves tooth enamel and leaves behind unwanted, chalky white spots that can appear permanently on the teeth.

The common causes of dental white spots include bacterial overgrowth in the mouth; excessive fluoride intake such as drinking too much fluoridated water or taking certain medications; insufficient calcium in diet; and demineralization due to acid buildup around braces. If not treated dental white spots will lead to dental decay which requires restorative procedures. Treating dental white spots usually requires the help of dentist. There is a need for new compositions and methods to treat and remove dental white spots.

### SUMMARY

The present invention provides bioactive regenerative materials and methods for making and using them. In one embodiment, the invention relates to the development of novel polydopamine (PDA) nanoparticles compositions, and the utilization of these compositions in dental applications.

In one aspect, a method is provided for remineralizing a hypocalcified tooth structure, the method comprises contacting said tooth structure with a composition comprising polydopamine nanoparticles. In some embodiments, the nanoparticles consist substantially of polydopamine. In some embodiments, the nanoparticles comprise polydopamine coating silica particles. In some embodiments, the nanoparticles comprise polydopamine coating polyethylene glycol nanoparticles.

In one embodiment, the polydopamine is polydopamine hydrochloride.

In some embodiments, the composition is a hydrogel. In some embodiments, the hydrogel comprises alginate. In some embodiments, the hydrogel comprises about 1 to about 5% polydopamine nanoparticles.

In some embodiments, the composition is a toothpaste. In some embodiments, the toothpaste comprises from about 1 to about 100 mg/mL polydopamine nanoparticles.

In one aspect, a composition is provided for remineralizing a hypocalcified tooth structure, the composition comprising polydopamine nanoparticles. In some embodiments, the nanoparticles consist substantially of polydopamine. In some embodiments, the nanoparticles comprise polydopamine coating silica particles. In some embodiments, the nanoparticles comprise polydopamine coating polyethylene glycol nanoparticles.

In one embodiment, the polydopamine is polydopamine hydrochloride.

In some embodiments, the composition is a hydrogel. In some embodiments, the hydrogel comprises alginate. In some embodiments, the hydrogel comprises about 1 to about 5% polydopamine nanoparticles.

In some embodiments, the composition is a toothpaste. In some embodiments, the toothpaste comprises from about 1 to about 100 mg/mL polydopamine nanoparticles.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1B shows scanning electron micrograph (SEM) of one embodiment of the nanoparticles disclosed herein. FIG. 1A shows a SEM of polydopamine nanoparticles. FIG. 1B shows a particle size distribution histogram as determined by SEM.

FIGS. 2A-2B shows Raman spectra (FIG. 2B) and mineralization images (FIG. 2A) of etched enamel by a toothpaste (TP) comprising polydopamine (CAT)-coated nanoparticles.

FIGS. 3A-3B shows Raman spectra (FIG. 3B) and mineralization images (FIG. 3A) of etched enamel incubated for 7 days with an alginate gel (Alg) comprising polydopamine (CAT)-coated nanoparticles.

FIGS. 4A-4B shows Raman spectra (FIG. 4B) and mineralization images (FIG. 4A) of etched dentin incubated for 7 days with an alginate gel (Alg) comprising polydopamine (CAT)-coated nanoparticles.

FIGS. 5A-5B shows Raman spectra (FIG. 5B) and mineralization images (FIG. 5A) of etched dentin incubated for 7 days with a toothpaste (TP) comprising polydopamine (CAT)-coated nanoparticles.

FIG. 6 shows scanning electron micrographs of mineralization of etched dentin incubated for 7 days with polydopamine (PDA or CAT)-coated nanoparticles.

FIG. 7 shows scanning electron micrographs of mineralization of etched enamel incubated for 7 days with polydopamine (CAT)-coated nanoparticles.

### DETAILED DESCRIPTION

In the present invention, a novel bioactive dental restorative material is introduced. This new dental material is based on polydopamine (PDA) nanoparticles with remineralization capability.

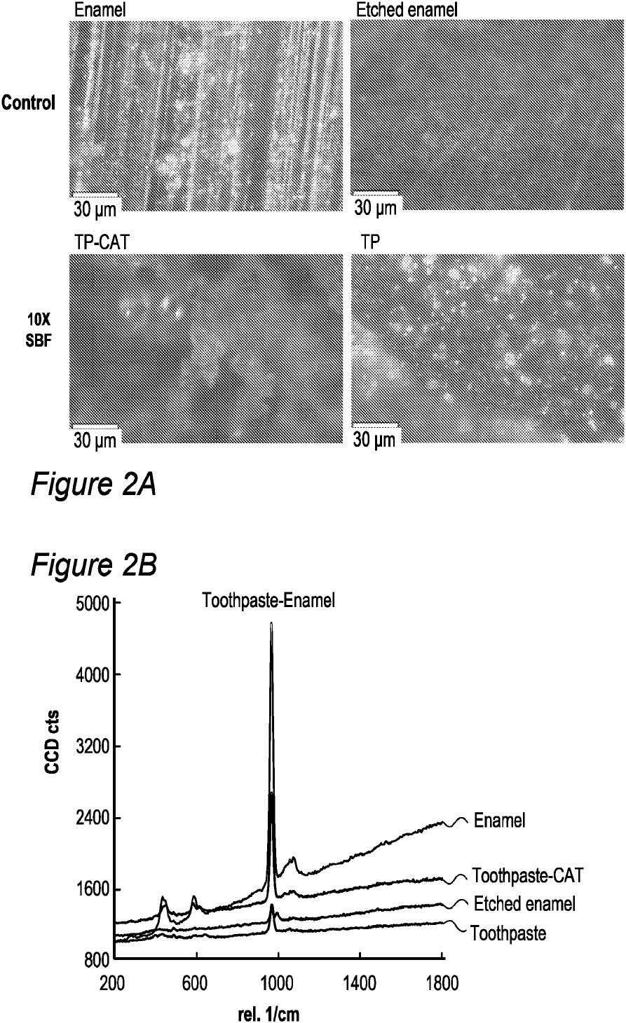

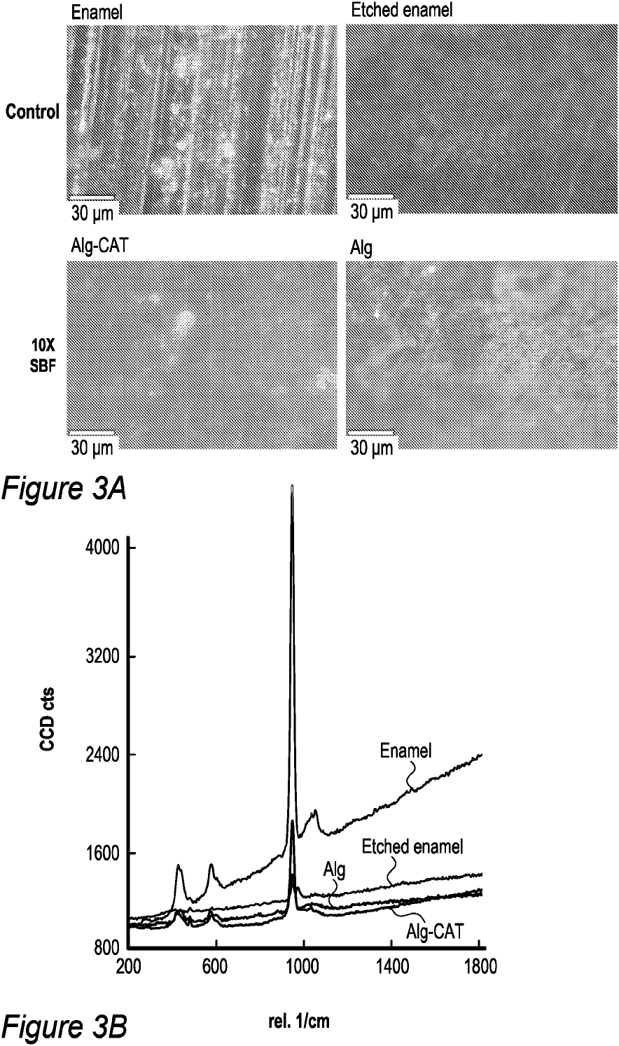

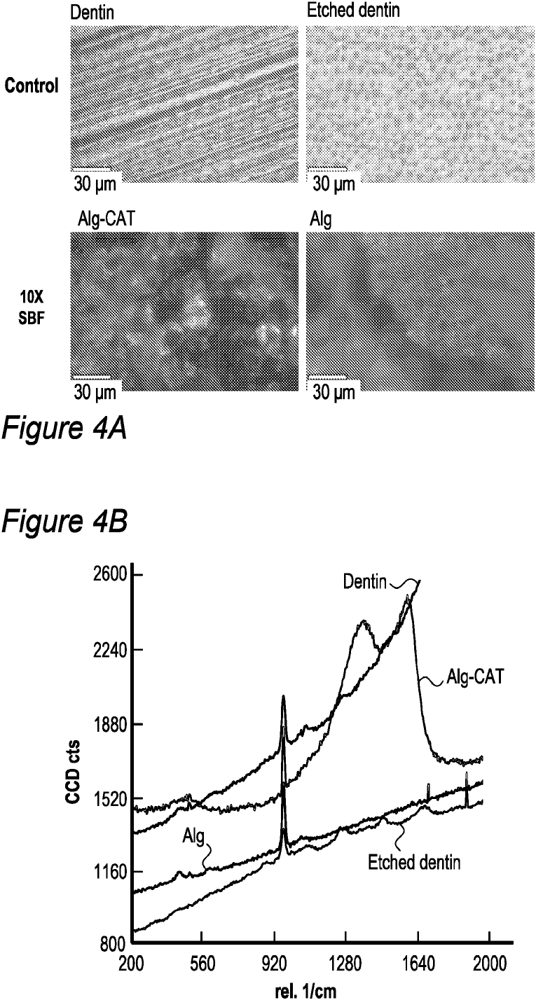

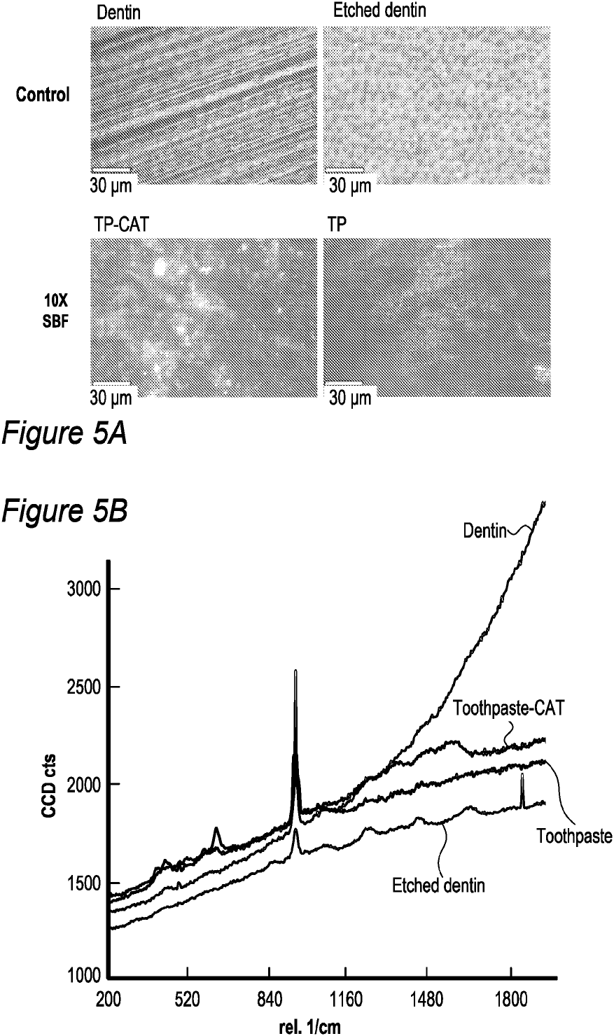

The presented data confirms the development of a new class of dental restorative materials with in-situ remineralization ability. In one embodiment, the material can be used as a direct dental restorative material. The disclosed material exhibits robust re-mineralization properties. In one illustration of the functional activities of this material, when the PDA nanoparticles were applied to the surface of etched dentin or enamel, a significant amount of mineralization has been observed (FIGS. 2 and 3) or with dentin (FIGS. 4 and 5) Therefore, this system can be used, for example, as novel treatment modality for tooth sensitivity or mineralization of tooth white spots.

In one embodiment, the present invention relates to synthesis of polydopamine (PDA) nanoparticles and their novel applications in dentistry. These novel PDA nanoparticles can regenerate a layer of hydroxyapatite on the surface of the tooth. In one embodiment, the PDA nanoparticles can be used as over-the-counter product against enamel white spots (pre-carious lesions) and dentin sensitivity. For example, a patient is able to use pre-fabricated trays containing the PDA nanoparticles disclosed herein intraorally similar to a bleaching tray or a nightguard. In one embodiment a toothpaste or gel is provided for brushing the teeth to treat white spots and sensitivity.

### Methods of Making

In one embodiment, the present disclosure provides a process for making polydopamine nanoparticles. In one embodiment, fabricating polydopamine nanoparticles with wide range of sizes (50-400 nm) is provided. As will be seen in the examples, the time of reaction, dopamine concentration, pH, and presence of oxidants can affect the resultant nanoparticles. The method for forming the dispersion will affect the resulting nanoparticles. In one embodiment, TRIS buffer (10-100 mM) is used as the reaction buffer and range of basic pHs (8-10.5) are used to form polydopamine nanoparticles. In some non-limiting embodiments, dopamine concentrations may vary from 0.5-10 mg/ml. Particles formed under the gentle shaking (150 rpm), and an Amicon Ultra-15 Centrifugal Filter (MWCO 100,000 Da) was used to wash the particles and make them concentrated before use. Optimized processing conditions resulted in monodisperse PDA nanoparticles with average diameter of 116±9 nm. This is a non-limiting example of one method for making the polydopamine nanoparticles as embodied herein.

In some embodiments, the polydopamine may be coated on nanoparticles. In some embodiments, the polydopamine may be coated on silica nanoparticles. In some embodiments, the polydopamine may be coated on polyethylene glycol (PEG) nanoparticles. In some embodiments, the polydopamine may be coated on titanium dioxide nanoparticles. In some embodiments, polydopamine at a concentration of 2 mg/mL is coated on 5 mg/mL silica particles, in a buffer and under other conditions as described herein. In some embodiments, mesoporous silica is used. While polydopamine can form nanoparticles without other components, in some embodiments a nanoparticulate material such as silica is used.

In some embodiments, a toothpaste, gel, or other formulation for application to the teeth contains the polydopamine nanoparticles and no other form of polydopamine.

In one embodiment, the buffer is mM tris(hydroxymethyl)aminomethane (TRIS) buffer. In one embodiment, the pH of the buffer is 8.5. In one embodiment, the dispersion is formed or the formation of the dispersion is facilitated by the use of vortexing. In one embodiment, ultrasonication is conducted at room temperature. In one embodiment, ultrasonication is used for 10 min or from 30 sec to 20 min. In one embodiment, stirring is applied for 4-16 h or for 1 h to 24 h. In one embodiment, stirring is conducted at room temperature. In one embodiment, stirring is conducted in the dark. In one embodiment, the nanoparticles were collected by centrifugation. In one embodiment, centrifugation is performed at 300 × g for 5 min. In one embodiment, the collected nanoparticles are washed with distilled water. In one embodiment, the collected nanoparticles are washed three times with milli-Q® water (>15 MΩ). In one embodiment, the collected nanoparticles are dried by a vacuum oven. Such nanoparticles substantially comprise only polydopamine.

In one embodiment the above process is scaled up for commercial manufacture of PDA nanoparticles and incorporation into toothpastes, gels, or other dentifrices for professional or personal use.

In some embodiments, in the process for making the polydopamine nanoparticles, silica particles are used. In such nanoparticles, polydopamine and silica are substantially the only components of the nanoparticles. In some embodiments the weight ratio of silica particles to polydopamine that are mixed to form the coated particles ranges between 20:1 and 1:20, between 1:1 and 100:1, between 50:1 and 10:1, between 100:1 and 1:1, between 1000:1 and 1:1, between 1000:1 and 1:10, between 10,000:1 and 1:1, between 20:1 and 1:1, or between 50:1 and 1:1 etc. Such silica particles have a large surface area for adsorption of polydopamine. Silica particles may be obtained from Sigma-Aldrich, such as silicon dioxide nanopowder, surface area 175-225 m²/g; monodisperse silicon dioxide, non-porous 150 nm nanoparticles, monodisperse silicon dioxide, non-porous 200 nm nanoparticles; monodisperse silicon dioxide, non-porous, 500 nm; and mesoporous silicon dioxide, 200 nm particle size.

Other nanoparticulate materials may be used similarly to silica in making the PDA nanoparticles embodied herein.

### Polydopamine Compositions

As it is generally understood in the art, nanoparticles are ultrafine particles with dimensions measured in nanometers (nm; 1 nm = 10⁻⁹ meter). Nanoparticles exist in the natural world and are also created as a result of human activities. Because of their submicroscopic size, nanoparticles have unique material characteristics, and manufactured nanoparticles may find practical applications in a variety of areas, including medicine, engineering, catalysis, and environmental remediation.

Nanoparticle has been defined as a discrete nano-object where all three Cartesian dimensions are less than about 100 nm. In another definition, nanoparticle is defined as a nano-object where only one of its characteristic dimensions is in the range of 1-100 nm, even if its other dimensions are outside that range. The lower limit of 1 nm is used because atomic bond lengths are reached at 0.1 nm. The size range—from 1 to 100 nm—overlaps considerably with that previously assigned to the field of colloid science—from 1 to 1,000 nm. Thus, it is not uncommon to find literature that refers to nanoparticles and colloidal particles in equal terms. The difference is essentially semantic for particles below 100 nm in size. In one embodiment the nanoparticles are 1 nm to less than about 150 nm. In one embodiment the nanoparticles are 1 nm to less than about 200 nm. In one embodiment the nanoparticles are 1 nm to less than about 250 nm. In one embodiment the nanoparticles are 1 nm to less than about 300 nm. In one embodiment the nanoparticles are 1 nm to less than about 350 nm. In one embodiment the nanoparticles are 1 nm to less than about 400 nm. In one embodiment the nanoparticles are 1 nm to less than about 450 nm. In one embodiment the nanoparticles are 1 nm to less than about 500 nm. In one embodiment the nanoparticles are about 50 nm to about 400 nm. In one embodiment the nanoparticles are about 10 nm to about 400 nm. In one embodiment the nanoparticles are about 50 nm to about 400 nm. In one embodiment the nanoparticles are about 50 nm to about 150 nm. In one embodiment the nanoparticles are about 80 nm to about 150 nm. In one embodiment the nanoparticles are about 120 nm. In one embodiment the nanoparticles are about 100 nm to about 130 nm. In one embodiment, polydopamine nanoparticles are provided with an average diameter of 116±9 nm. In one embodiment, the nanoparticles are monodisperse. In one embodiment the nanoparticles are substantially a homogeneous population. In one embodiment the nanoparticles are predominantly a homogeneous population.

Nanoparticles can be classified according to their size, shape, and material properties. Some classifications distinguish between organic and inorganic nanoparticles; the first group includes dendrimers, liposomes, and polymeric nanoparticles, while the latter includes fullerenes, quantum dots, and gold nanoparticles. Other classifications divide nanoparticles according to whether they are carbon-based, ceramic, semiconducting, or polymeric. The way in which nanoparticles are classified typically depends on their application, such as in diagnosis or therapy versus basic research, or may be related to the way in which they were produced.

In one embodiment, the nanoparticles of this invention are ball-shaped or circular shaped and their size is defined by their diameter. However, nanoparticles disclosed herein can be of any shape including rod-like particles, other elongated particles, non-symmetric ball-shaped particles, polyhedral, rectangular, cube-shaped, oval-shaped, or particles of any other form, including symmetric, non-symmetric or partially symmetric particles, particles with smooth surface, particles with rough surface or any combination thereof. In one embodiment, the nanoparticles comprise polydopamine coated on silica nanoparticles.

In the embodiments herein, dopamine refers to the catecholamine (CAT) dopamine. Dopamine has the chemical name 3,4-dihydroxyphenethylamine (C₈H₁₁NO₂); synonyms and abbreviations include DA, 2-(3,4-dihydroxyphenyl)ethylamine, 3,4-dihydroxyphenethylamine, 3-hydroxytyramine, and oxytyramine. The CAS registry number is 51-61-6 and that of the hydrochloride is 62-31-7. It has a molecular mass of 153.181 g/mol. Polydopamine (PDA) hydrochloride is a polymer of polydopamine, or polymerized dopamine, which forms by a spontaneous oxidation reaction, and is formally a type of melanin. Synthesis usually involves reaction of dopamine hydrochloride with Tris as a base in water. In some embodiments, polydopamine nanoparticles are the only form of polydopamine in the formulations embodied herein.

In some embodiments, an alternate catecholamine than dopamine may be used, such as another polyphenolic biomolecule. In one embodiment, the polyphenolic biomolecule is tannin. Non-limiting examples of tannins include gallic acid, phloroglucinol and flavan-3-ol. Such compounds or polymers thereof may be used in the various embodiments herein.

In some embodiments, the polydopamine is coated on nanoparticles, which nanoparticles may be silica (silicon dioxide) particles, titanium dioxide nanoparticles, polyethylene glycol nanoparticles, by way of non-limiting examples.

While dopamine as the starting material and polydopamine as the polymeric product are named and described herein, it should be noted that dopamine is a charged molecule and is available commercially and purchased most often as the hydrochloride salt (molecular mass 189.64 g/mol).

As noted above, in one embodiment, nanoparticles consisting of polydopamine are provided. In one embodiment, nanoparticles of around average diameter of 116 nm are provided.

In one embodiment, nanoparticles comprising polydopamine on a particulate material such as but not limited to silica is embodied herein. In one embodiment, the thickness of the PDA coating on nanoparticles of this invention ranges from 5 nm to 50 nm. In other embodiments, the thickness of the PDA coating ranges from 1 nm to 100 nm, 5 nm to 100 nm, 1 nm to 1 micron, or 1 nm to 100 microns.

In one embodiment, the nanoparticles are fully-coated by PDA. In another embodiment, the nanoparticles are partially coated by PDA. In another embodiment, clusters or aggregates of nanoparticles are coated (fully or partially) by PDA. Embodiments of this invention include collections of nanoparticles coated by PDA wherein the nanoparticles are fully coated, partially coated or wherein some nanoparticles are fully coated while others are partially coated. In one embodiment, nanoparticles fully or partially coated with PDA are mixed with particles that are not coated by PDA to form compositions of nanoparticles of this invention. In other embodiments, the PDA coated particles (fully or partially or combinations thereof) are used in compositions of this invention without additional non-PDA-coated particles. In some embodiments, the nanoparticles are more than 50% coated. In other embodiments, the nanoparticles are less than 50% coated by PDA.

In some embodiments, nanoparticles consisting of PDA are mixed with nanoparticles coated with PDA. In some embodiments the nanoparticles can be from about 1% coated with PDA to 100% coated with PDA.

### Toothpaste and Gel Formulations

Formulations for application of the PDA nanoparticles to the teeth are embraced herein. In some embodiments, the PDA nanoparticles are formulated in a dentifrice such as but not limited to a toothpaste, a tooth gel, or a tooth powder. Such dentifrices are for application to the teeth by the individual or by the dental professional.

In some embodiments, the PDA nanoparticles are formulated in a toothpaste. In one non-limiting example, the toothpaste comprises any of various toothpaste formulations well known to one of skill in the art, including but not limited to over-the-counter and professional (prescription) toothpastes. In one embodiment, the toothpaste comprises a combination of tricalcium phosphate, sodium fluoride, water, non-crystallizing sorbitol solution, synthetic amorphous precipitated silica, glycerin, amorphous silica, polyethylene-polypropylene glycol, polyethylene glycol, sodium saccharin, titanium dioxide, flavorings mixture, sodium carboxymethyl cellulose, and sodium lauryl sulfate, or any combination of any of the foregoing components.

In some embodiments, the polydopamine nanoparticles described herein are provided in a gel (hydrogel) formulation, for direct application to the teeth or for further formulation in a toothpaste. In one embodiment, the gel is a sodium alginate hydrogel. In one embodiment the sodium alginate hydrogel is made by mixing alginate hydrogel 1.2% solution containing PDA at different loadings and adding calcium ions from CaCl₂ solution. In one embodiment the sodium alginate gel comprises about 1% to about 10% PDA nanoparticles. In one embodiment the sodium alginate gel comprises about 1.5% PDA nanoparticles. In some embodiment the concentration of PDA nanoparticles in a gel is from about 1% to about 5%. As noted herein, the gel comprising PDA nanoparticles may be provided for direct application to the teeth, such as in a dental tray, or may be formulation in a toothpaste. In one embodiment, use of a dental tray allows for a longer application of the PDA dentifrice to the teeth than would occur during routing brushing with a toothpaste.

In some embodiments, the hydrogel is a carboxymethyl cellulose hydrogel. In some embodiments the hydrogel is a polyvinylpyrrolidone hydrogel. In some embodiments the hydrogel is an acrylamide/acrylic acid co-polymer hydrogel. In some embodiments the hydrogel is a poly(2-hydroxyethyl methacrylate) hydrogel. In some embodiments, the hydrogel is a methacrylic acid hydrogel. In some embodiments, the hydrogel is a polyethylene glycol methacrylate hydrogel. In some embodiments, the hydrogel is an acrylic acid/acrylamide hydrogel. In some embodiments, the hydrogel is a carboxymethyl cellulose hydrogel. In some embodiments, the hydrogel is a chitosan hydrogel. The foregoing are merely non-limiting examples of hydrogels useful for providing a polydopamine composition for the purposes herein.

In one embodiment, PDA silica nanoparticles are mixed (1.5% (w/v)) with alginate hydrogel and formulated in 3 M CLINPRO tooth crème at a final concentration of 100 mg/mL. In some embodiments, the concentration of polydopamine in a formulation is 100 mg/mL, though this may vary depending on the formulation, application instructions, stability, storage conditions, and other factors. In some embodiments the concentration of PDA nanoparticles can be from about 1 to about 200 mg/mL in the toothpaste. In some embodiments the concentration of PDA nanoparticles can be from about 1 to about 100 mg/mL in the toothpaste.

### Methods of Use

In some embodiments, the present disclosure provides a method of using the nanoparticles described herein to generate a layer of hydroxyapatite at the surface of a tooth, the method comprising applying a composition comprising the nanoparticles disclosed herein to a surface of a tooth. In some embodiments, this method is used for treatment of incipient caries (white spots) on tooth structure. White spots are hypocalcified structures that are not yet decayed. In one embodiment, the composition is in the form of a hydrogel. In another embodiment, the composition is in the form of toothpaste. The composition disclosed herein would extract calcium and phosphate from the patient’s saliva and start remineralization. In one embodiment, the composition application step is being conducted by a physician or by a patient. In one embodiment, the composition can be supplied as an over-the-counter (OTC) product. In one embodiment, the OTC product comprises a pre-loaded tray with PDA nanoparticles or PDA-coated nanoparticles, in a suitable formulation such as a hydrogel.

In some embodiments, the compositions disclosed herein facilitate hydroxyapatite remineralization, for example, after 7 days following daily application of the composition to a surface of a tooth, hydroxyapatite remineralization is observed. Typically, this hydroxyapatite remineralization is at least 10% (e.g. at least 30%, 50%, 70%, 90%, or 100%) greater than hydroxyapatite remineralization observed with a control/comparative composition without the nanoparticles disclosed herein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

### Example 1

### Fabrication of Polydopamine (PDA) Nanoparticles

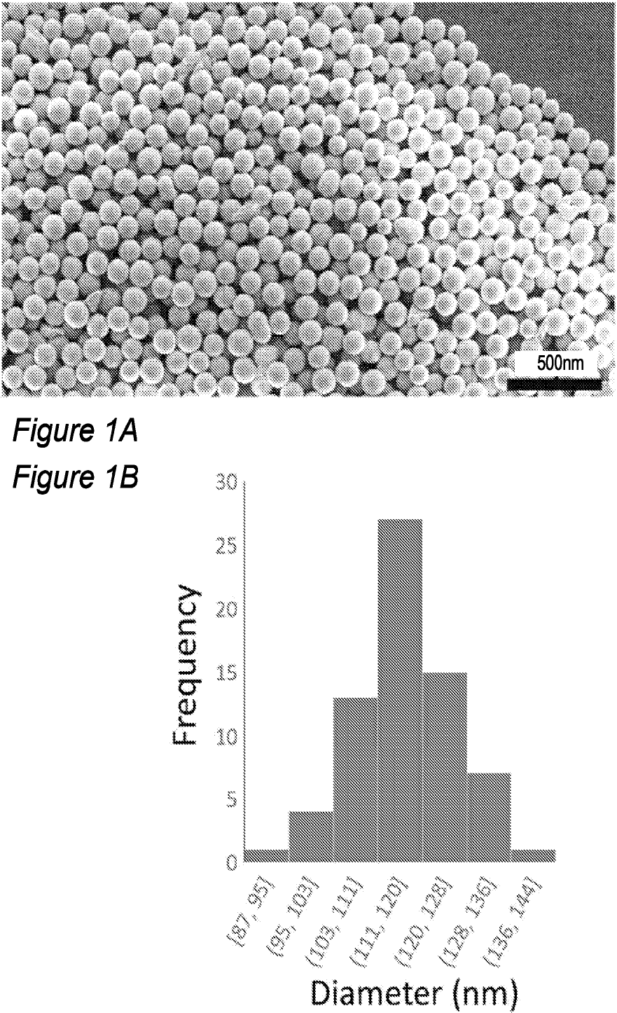

It is generally known in the art that polydopamine nanoparticles can be made under various conditions, for example, time of reaction, dopamine concentration, pH, and presence of oxidants can affect the resulted nanoparticles. The present example discloses processes of fabricating polydopamine nanoparticles with wide range of sizes (e.g. 50-400 nm). Tris buffer (10-100 mM) may be used as reaction buffer and ranges of basic pHs (8-10.5) were used to form polydopamine nanoparticles. Polydopamine concentrations were also varied from 0.5-10 mg/ml in the current study. Particles formed under gentle shaking (150 rpm) were washed with Amicon Ultra-15 centrifugal filter (MWCO 100,000 Da) and concentrated before use. Optimized processing conditions resulted in monodisperse PDA nanoparticles with average diameter of 116±9 nm (see FIG. 1B).

### Example 2

### Fabrication of Polydopamine (PDA)-coated Nanoparticles

In one embodiment, catecholamine (dopamine) hydrochloride powder (2 mg/ml) was mixed with 10 mM Tris-HCl, pH 8.5. Silica particles (Sigma-Aldrich catalogue no. 904449, monodisperse, non-porous silicon dioxide, 500 nm; 5 mg/ml) were added to the mixture and rotated for about 16 hrs. The mixture was then washed multiple times and dried. The dried PDA-coated particles powder was mixed with alginate hydrogel (1.5% wt/v) or toothpaste (e.g. 3 M CLINPRO Tooth crème) at a final polydopamine concentration of 100 mg/ml.

### Example 3

### Remineralization Properties

The present example examines the effects of applying PDA-coated nanoparticles on white spots (dental caries in their initial stage).

Compositions comprising PDA-coated nanoparticles were formulated as hydrogel (1.5% wt/vol PDA-coated silica) or toothpaste (100 mg/mL PDA-coated silica) as described in Example 2 above. Dentin/enamel slices were etched right before experiments. In the hydrogel group, the nanoparticles mixture was mixed with 25 mM calcium sulfate and applied on the tooth slices. Alginate without PDA-coated nanoparticles served as the control. In the toothpaste group, all dentin and enamel slices were brushed with the toothpaste mixture 40 times, twice a day. The toothpaste without PDA-coated nanoparticles served as the control. The dentin/enamel slices were incubated in 10X artificial saliva (10X SBF; SBF: 0.2 mM MgCl₂, 1 mM CaCl₂ H₂O, 20 mM HEPES buffer, 4 mM KH₂PO₄, 16 mM KC1, 4.5 mM NH4C1, 300 p.p.m. NaF, pH 7.0, adjusted with 1 M NaOH) for 7 days. The solution was replaced with freshly prepared solution every other day. The samples were rinsed and dried before performing SEM or Raman spectroscopy.

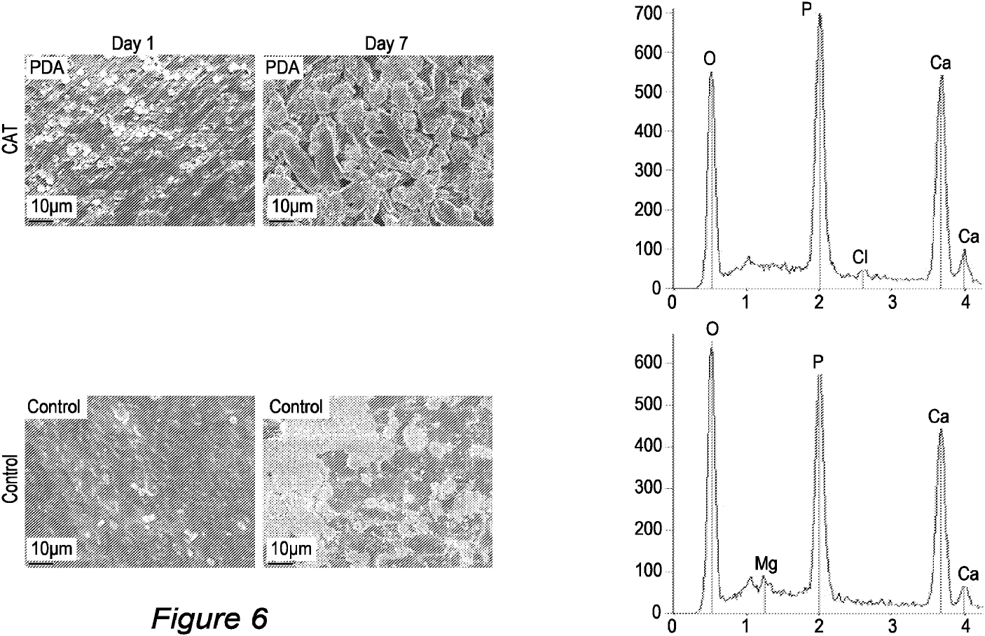

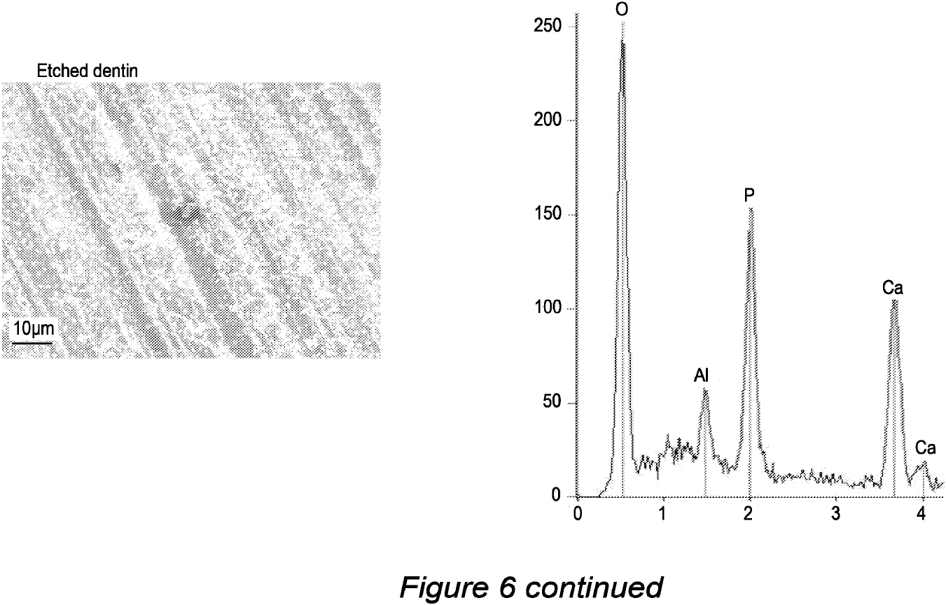

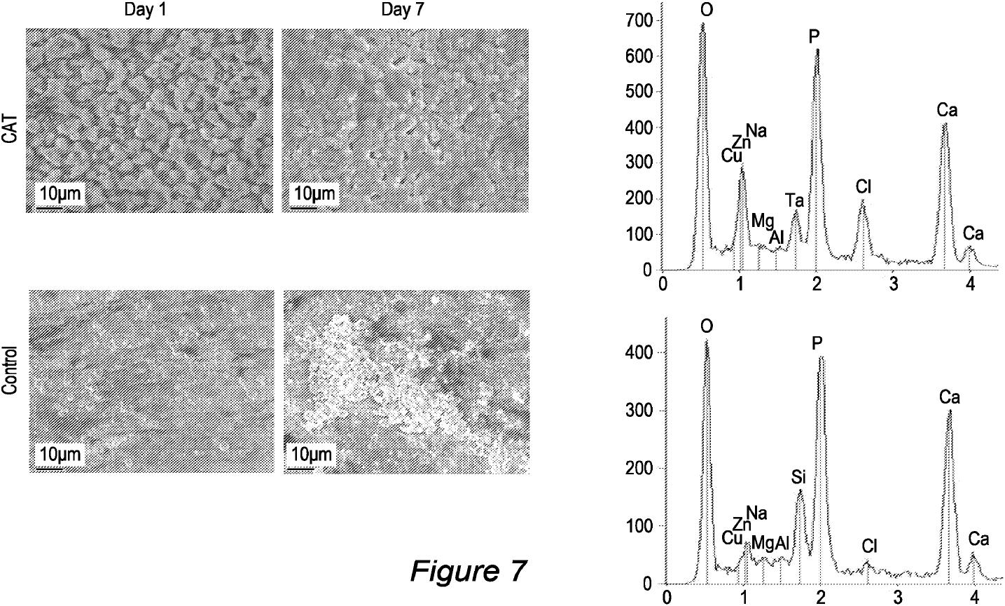

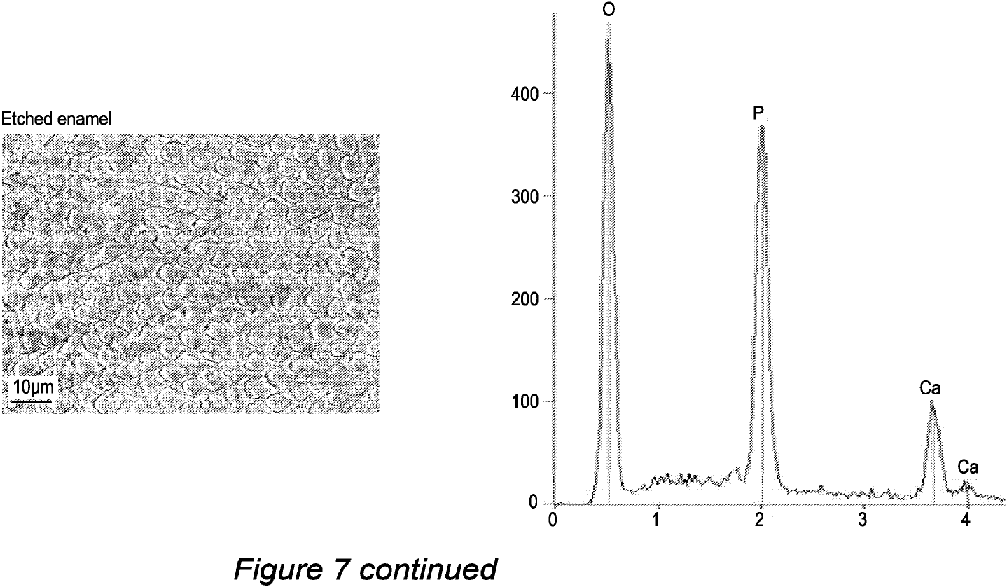

Comparison of the enamel surface with white spot (demineralized surface) after application of control and compositions comprising PDA-coated nanoparticles was performed. FIGS. 2A-2B shows the results of remineralization using etched enamel and toothpaste. FIGS. 3A-3B shows the results of remineralization using etched enamel and alginate hydrogel. FIGS. 4A-4B shows the results of remineralization using etched dentin and alginate hydrogel. FIGS. 5A-5B shows the results of remineralization using etched dentin and toothpaste. FIG. 6 shows scanning electron micrographs of the mineralized dentin after application of the PDA containing toothpaste1 and 7 days. FIG. 7 shows a scanning electron micrograph of the mineralized enamel after application of the PDA containing toothpaste after 1 and 7 days.

### Example 4

### Remineralization Properties

A similar experiment as described in Example 3 is carried out using PDA nanoparticles prepared by the method of Example 1, wherein no silica particles are used in the preparation of the nanoparticles. Similar results as described in Example 3 are obtained, showing that the gel or toothpaste containing PDA nanoparticles remineralizes etched dentin and enamel.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for remineralizing a hypocalcified tooth structure, said method comprises contacting said tooth structure with a composition comprising polydopamine nanoparticles.

2. The method of claim 1, wherein the nanoparticles consist substantially of polydopamine.

3. The method of claim 1, wherein said nanoparticles comprise polydopamine coating silica particles or titanium dioxide particles.

4. The method of claim 1, wherein said nanoparticles comprise polydopamine coating titanium dioxide particles.

5. The method of claim 1, wherein said nanoparticles comprise polydopamine coating polyethylene glycol nanoparticles.

6. The method of claim 1 wherein the polydopamine is polydopamine hydrochloride.

7. The method of claim 1, wherein said composition is a hydrogel.

8. The method of claim 7, wherein said hydrogel comprises alginate.

9. The method of claim 7, wherein the hydrogel comprises about 1 to about 5% polydopamine nanoparticles.

10. The method of claim 1, wherein said composition is a toothpaste.

11. The method of claim 10, wherein the toothpaste comprises from about 1 to about 100 mg/mL polydopamine nanoparticles.

12. A composition for remineralizing a hypocalcified tooth structure, said composition comprising polydopamine nanoparticles.

13. The composition of claim 12, wherein the nanoparticles consist substantially of polydopamine.

14. The composition of claim 12, wherein said nanoparticles comprise polydopamine coating silica particles.

15. The composition of claim 12, wherein said nanoparticles comprise polydopamine coating titanium dioxide particles.

16. The composition of claim 12, wherein said nanoparticles comprise polydopamine coating polyethylene glycol nanoparticles.

17. The composition of claim 12 wherein the polydopamine is polydopamine hydrochloride.

18. The composition of claim 12, wherein said composition is a hydrogel.

19. The composition of claim 18, wherein said hydrogel comprises alginate.

20. The composition of claim 18, wherein the hydrogel comprises about 1 to about 5% polydopamine nanoparticles.

21. The composition of claim 12, wherein said composition is a toothpaste.

22. The composition of claim 21, wherein the toothpaste comprises from about 1 to about 100 mg/mL polydopamine nanoparticles.
